(19) United States
(12) Patent Application Publication
(10) Pub. No.: US 2004/0146477 A1
(43) Pub. Date: Jul. 29, 2004

(54) Cosmetic or pharmaceutical agent

(76) Inventors: Helmut Meffert, Ludwigshafen (DE); Axel Sanner, Frakenthal (DE); Kristin Tiefensee, Bad Duerkheim (DE); Stephan Kothrade, Limburgerhof (DE)

Correspondence Address: KEIL & WEINKAUF, 1350 CONNECTICUT AVENUE, N.W., WASHINGTON, DC, 20036, US

(21) Appl. No.: 10/467,946

(22) PCT Filed: Feb. 20, 2002
(86) PCT No.: PCT/EP02/01802

(30) Foreign Application Priority Data
Feb. 21, 2001 (DE) 101 08 387.4

(51) Int. Cl. A61K 7/06; A61K 7/11

(52) U.S. Cl. 424/70.15

(57) ABSTRACT

The present invention relates to cosmetic or pharmaceutical compositions having a liquid to gel-like consistency which comprise at least one copolymer which comprises at least one N-vinyllactam and at least one ethylenically unsaturated monomer having a longer-chain alkyl or alkylene radical in copolymerized form. The invention also relates to the use of such copolymers in cosmetic or pharmaceutical compositions.

The present invention relates to cosmetic or pharmaceutical compositions having a liquid to gel-like consistency which comprise at least one copolymer which comprises at least one N-vinyllactam and at least one ethylenically unsaturated monomer having a longer-chain alkyl or alkylene radical in copolymerized form. The invention further relates to the use of such copolymers in cosmetic or pharmaceutical compositions.

Cosmetic products for the treatment of skin and hair are subject to a large number of requirements. Very generally, they should have good properties both with regard to their handlability and applicability during preparation and for the end user, and also with regard to the effect achieved therewith. For example, skin and hair cosmetic and dermatological compositions should be present in formulations which permit the active ingredients present to readily develop their action at the site of action. They should be well tolerated by the skin and, in the case of skin or hair, exhibit further advantageous effects such as, for example, cleansing, care, protection, moisturizing, conditioning etc. Said advantageous effects for the user are usually accompanied by a positive subjective feeling of having overall healthy and cared-for skin or hair.

To prepare cosmetic and pharmaceutical products, use is frequently made of hydrophobic substances with a nonsolid consistency, such as oils, fats, waxes etc., which are soluble in water only to a minor degree, if at all. Hydrophobic substances, such as oils which are liquid at room temperature, are often impractical and difficult to handle and apply. For this reason, cosmetic and pharmaceutical compositions are frequently subject to special requirements with regard to their rheological properties. They can often only be formulated in the desired application form, such as, for example, gels, creams or emulsions, using additives, so-called thickeners. Examples of customary low molecular weight thickeners, are, for example, the alkali metal and aluminum salts of fatty acids, fatty alcohols or waxes. However, the use of known thickeners is frequently associated with disadvantages, depending on the field of use of the preparation to be thickened. For example, either the thickening action of the thickeners may be unsatisfactory, their use may be undesired or their incorporation into the preparation to be thickened is hindered or completely impossible because of their incompatibility with the water-insoluble compound to be thickened. A proportion of thickener of at least 50% by weight, based on the total formulation, is often required to establish the desired viscosity. In addition, many traditional thickeners, primarily waxes, produce an unpleasant sticky feel on the skin.

Cosmetic and pharmaceutical products which, in addition to or instead of a hydrophobic component, have at least one hydrophilic component and/or water are also subject to special requirements. These include, for example, in turn the rheological properties and the possibility of formulating stable W/O and O/W emulsions. In the case of such products in the form of gels, the consumer increasingly prefers clear, opaque formulations.

Another current requirement of cosmetic and pharmaceutical compositions is to keep the proportion of nonactive substances, such as thickeners, as low as possible. There is therefore a need for additives or auxiliaries which develop their action even in small amounts and/or which as far as possible have a positive influence on two or more of the abovementioned product properties.

It is known to use polymers to modify the rheological properties of essentially water-insoluble liquids. These have the advantage that, provided they are soluble in the hydrophobic substance, they generally permit the viscosity to be adjusted as a function of their molecular weight.

DE-A-2833711 describes sunscreen formulations which comprise oil-soluble acrylate polymers to increase the water resistance.

U.S. Pat. No. 5,318,995 describes a cosmetic composition which comprises a W/O emulsion which comprises a thickener which is a copolymer having units derived from (meth)acrylates or (meth)acrylamides, and units having terminal carboxylic acid or sulfonic acid groups.

WO-A-93/01797 describes a cosmetic composition comprising an oil phase which comprises a combination of two copolymers as thickener. Here, the first copolymer has units derived from lipophilic monomers and from hydrophilic monomers, the latter comprising at least one carboxylic acid and/or sulfonic acid group. The second copolymer has units derived from at least one lipophilic and from at least one hydrophilic monomer, the latter having at least one amine, amide, alcohol or ether group.

WO-A-98/30194 describes a composition comprising an oil phase and a polymer thickener which has hydrophobic functionalities which are suitable for imparting at least partial solubility in the oil, and which has hydrophilic functionalities which bring about the thickening, these hydrophilic functionalities being introduced by monomers chosen from C₃-C₆-α,β-ethylenically unsaturated monocarboxylic acids, C₄-C₆-α,β-ethylenically unsaturated dicarboxylic acids and monoesters and monoamides thereof.

DE-A-19627204 describes cosmetic or pharmaceutical compositions with high water resistance which comprise at least one polymer built up from at least 20% by weight of (meth)acrylic esters. These polymers comprise at most 30% by weight of water-soluble monomers.

DE-A-19815127 describes a composition comprising at least one water-insoluble ingredient and at least one copolymer of

A) 5 to 99% by weight of at least one N-vinylcarboxamide, which is not a lactam,

B) 1 to 95% by weight of at least one hydrophobic monomer and optionally

C) 0 to 94% by weight of another monomer which may, inter alia, be an α,β-ethylenically unsaturated carboxylic acid or an N-vinyllactam.

DE-A-19753298 describes a process for the preparation of solid dosage forms by mixing at least one polymeric binder and at least one active ingredient to form a plastic mixture, and shaping, the polymeric binder used being a copolymer of an N-vinyllactam and a monomer containing a hydrophobic radical which is copolymerizable with the N-vinyllactam, and the dosage forms obtained by this process.

EP-A-0953358 describes the use of copolymers comprising 50 to 99% by weight of at least one N-vinyllactam or N-vinylamine and 1 to 50% by weight of at least one monomer containing a hydrophobic group as matrix for the preparation of solid pharmaceutical and cosmetic administration forms.

The unpublished German patent applications P 100 22 247.1 and P 199 41 365.7 describe the use of polyurethanes for modifying the Theological properties of hydrophobic liquids.

It is an object of the present invention to provide cosmetic or pharmaceutical compositions with a liquid to gel-like consistency which have good performance properties. They should preferably be suitable for the cosmetic and dermatological treatment of skin and/or hair. In particular, it should be possible to adjust their rheological properties and consistency within as wide a range as possible.

We have found that this object is achieved by a cosmetic composition which comprises at least one copolymer which comprises 60 to 99% by weight of at least one hydrophilic monomer and, of this, at least 65% by weight comprises at least one N-vinyllactam, and 1 to 40% by weight of at least one hydrophobic monomer, in copolymerized form.

The present invention therefore provides a cosmetic or pharmaceutical composition with a liquid to gel-like consistency, comprising:

A) at least one copolymer which comprises, based on the total amount of monomer,

a) 60 to 99% by weight of at least one hydrophilic monomer and, of this, at least 65% by weight comprises at least one N-vinyllactam, and

b) 1 to 40% by weight of at least one hydrophobic monomer chosen from

b1) compounds of the formula I

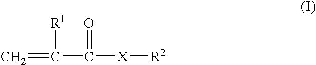

in which

R¹ is H, methyl or ethyl,

X is O, NH or NR³,

R² is C₈-C₃₀-alkyl, C₈-C₃₀-alkenyl, cycloalkyl-C₂-C₂₂-alkyl, cycloalkyl-C₂-C₂₂-alkenyl, aryl-C₂-C₂₂-alkyl or aryl-C₂-C₂₂-alkenyl, and

R³ is C₁-C₇-alkyl, cycloalkyl, aryl or one of the meanings given for R²,

b2) vinyl esters of aliphatic C₈-C₃₀-carboxylic acids,

b3) C₈-C₃₀-alkyl vinyl ethers

and mixtures thereof,

in copolymerized form, and

B) at least one cosmetically acceptable additive.

For the purposes of the present invention, the expressions C₈-C₃₀-alkyl, C₂-C₂₂-alkyl and C₁-C₇-alkyl include straight-chain and branched alkyl groups. Suitable short-chain alkyl groups are, for example, straight-chain or branched C₁-C₇-alkyl, preferably C₁-C₆-alkyl and particularly preferably C₁-C₄-alkyl groups. These include, in particular, methyl, ethyl, propyl, isopropyl, n-butyl, 2-butyl, sec-butyl; tert-butyl, n-pentyl, 2-pentyl, 2-methylbutyl, 3-methylbutyl, 1,2-dimethylpropyl, 1,1-dimethylpropyl, 2,2-dimethylpropyl, 1-ethylpropyl, n-hexyl, 2-hexyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl and C₈-C₃₀-alkenyl groups are straight-chain and branched alkyl and alkenyl groups. These are preferably predominantly linear alkyl radicals, as also occur in natural or synthetic fatty acids and fatty alcohols, and oxo alcohols, which may additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene) etc.

Cycloalkyl is preferably C₅-C₈-cycloalkyl, such as cyclopentyl, cyclohexyl, cycloheptyl or cyclooctyl.

Aryl is preferably phenyl, tolyl, xylyl or naphthyl.

Compounds which can be derived from acrylic acid and methacrylic acid are sometimes referred to in abbreviated form by adding the syllable “(meth)” to the compound derived from acrylic acid.

The compositions according to the invention have a liquid to gel-like consistency under standard conditions (20° C.). This is generally understood as meaning rheological properties which range from thin-liquid via pasty/ointment-like to gel-like. The viscosity of the liquid compositions is preferably in a range from about 1 to 6000 mPas. “Gel like consistency” is shown by compositions which have a higher viscosity than a liquid and which are self-supporting, i.e. they retain a shape given to them without a shape-stabilizing coating. In contrast to solid formulations, however, gel-like formulations can be readily deformed under the application of gravitational forces. The viscosity of the gel-like compositions is preferably in a range greater than 600 to about 60 000 mPas.

The copolymer A) comprises 60 to 99% by weight, preferably 65 to 95% by weight, of at least one hydrophilic monomer in copolymerized form. For the purposes of the present invention, the term “hydrophilic monomer” refers to a monomer which has a solubility in water of at least 10 g/l, preferably at least 50 g/l, at 20° C. At least 65% by weight of the compounds of component a) are chosen from N-vinyllactams, preferably from N-vinylpyrrolidone, N-vinylpiperidone, N-vinylcaprolactam and mixtures thereof.

The component a) can, in addition to the N-vinyllactams, have at least one other hydrophilic monomer. The additional hydrophilic monomers of component a) preferably comprise at least one α,β-ethylenically unsaturated mono- and/or dicarboxylic acid. Particular preference is given to monoethylenically unsaturated-carboxylic acids having 3 to 8 carbon atoms, such as acrylic acid, methacrylic acid, dimethacrylic acid, ethacrylic acid, maleic acid, citraconic acid, methylenemalonic acid, allylacetic acid, vinylacetic, crotonic acid, fumaric acid, mesaconic acid, itaconic acid and mixtures thereof. In particular, acrylic acid, methacrylic acid, maleic acid and mixtures thereof and, in particular, acrylic acid are used as component a). The abovementioned carboxylic acids can be used in the form of the free acid, if present, the anhydrides or in partially or completely neutralized form. Bases which can be used for the neutralization of the carboxylic acids are alkali metal bases, such as sodium hydroxide solution, potassium hydroxide solution, sodium carbonate, sodium hydrogencarbonate, potassium carbonate or potassium hydrogencarbonate, alkaline earth metal bases, such as calcium hydroxide, calcium oxide, magnesium hydroxide or magnesium carbonate, ammonia and amines. Suitable amines are, for example, C₁-C₆-alkylamine, preferably n-propylamine and n-butylamine, dialkylamines, trialkylamines, preferably diethylpropylamine, dipropylmethylamine, triethylamine and triisopropylamine, aminoalcohols, preferably ethanolamine, methylethanolamine, ethylethanolamine, diethanolamine, triethanolamine, 2-amino-2-methyl-1-propanol, triisopropanolamine and morpholine, diethylaminepropylamine, diethylenetriamine, tetraethylenepentamine, glucamine, methylglucamine and mixtures thereof.

Further suitable hydrophilic comonomers a) are, for example, the esters of the abovementioned α,β-ethylenically unsaturated mono- and/or dicarboxylic acids with C₁-C₄-alcohols, such as methyl (meth)acrylate, ethyl (meth)acrylate, n-propyl (meth)acrylate, monomethyl maleate, dimethyl maleate, monoethyl maleate, diethyl maleate etc.

Suitable further comonomers a) are, for example, the esters of the abovementioned α,β-ethylenically unsaturated mono- and/or dicarboxylic acids with at least dihydric alcohols, preferably C₂-C₃₀-alkanediols, and alkoxylates thereof. These include, for example, 2-hydroxyethyl (meth)acrylate, 3-hydroxy-n-propyl (meth)acrylate, hydroxyisobutyl (meth)acrylate, 4-hydroxy-n-butyl (meth)acrylate, hydroxy-tert-butyl (meth)acrylate, 3-hydroxy-2-ethylhexyl (meth)acrylate etc.

Suitable further comonomers a) are the amides and nitriles of the abovementioned α,β-ethylenically unsaturated mono- and/or dicarboxylic acids, such as (meth)acrylamide, N,N-dimethyl(meth)acrylamide, N-tert-butyl(meth)acrylamide, (meth)acrylonitrile etc.

Suitable further comonomers a) are vinyl- and allyl-substituted heteroaromatic compounds, such as N-vinylimidazole, N-vinyl-2-methylimidazole, 2- and 4-vinylpyridine, -allylpyridine etc.

Suitable further comonomers a) are noncyclic N-vinylcarboxamides, such as N-vinylformamide, N-vinylacetamide, N-vinylpropionamide, vinylbutyramide etc.

Suitable further comonomers a) are (meth)acrylamidoglycolic acid, vinylsulfonic acid, (meth)allylsulfonic acid, styrenesulfonic acid, 3-sulfopropyl (meth)acrylate, (meth)acrylamidomethylpropanesulfonic acid, monomers containing phosphonic acid groups, such as vinylphosphonic acid, allylphosphonic acid and acrylamidomethanepropanephosphonic acid etc.

The abovementioned compounds of component a) can be used individually or in the form of mixtures.

The hydrophobic monomer component b) of copolymer A) preferably comprises at least one compound b1) of the formula I, as defined above. These include, preferably, the esters of acrylic acid, methacrylic acid or ethacrylic acid with a saturated or mono- or polyunsaturated C₈-C₁₈-alcohol, such as octyl (meth)acrylate, 2-ethylhexyl (meth)acrylate, nonyl (meth)acrylate, decycl (meth)acrylate, lauryl (meth)acrylate, pentadecyl (meth)acrylate, palmityl (meth)acrylate, arrachinyl (meth)acrylate, myristyl (meth)acrylate, cetyl (meth)acrylate, stearyl (meth)acrylate, lignocerenyl (meth)acrylate, cerotinyl (meth)acrylate, melissinyl (meth)acrylate, palmitoleinyl (meth)acrylate, oleyl (meth)acrylate, behenyl (meth)acrylate, cyclohexylethyl (meth)acrylate, cyclohexyl-tert-butyl (meth)acrylate etc.

Suitable monomers b1) are also the amides of acrylic acid, methacrylic acid or ethacrylic acid which have at least one radical R³ as defined above, on the amide nitrogen atom. These include, for example, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleinyl(meth)acrylamide, oleyl(meth)acrylamide, linolyl(meth)acrylamide, linolenyl(meth)acrylamide, stearyl(meth)acrylamide, lauryl(meth)acrylamide and mixtures thereof. Suitable monomers b1) are also the N-alkylamides, N,N′-dialkylamides, N-alkyldiamides and N,N′-dialkyldiamides of maleic acid.

Particularly preferred monomers b1) are 2-ethylhexyl (meth)acrylate, lauryl (meth)acrylate, myristyl (meth)acrylate, cetyl (meth)acrylate, stearyl (meth)acrylate, oleyl (meth)acrylate, behenyl (meth)acrylate and mixtures thereof.

Preferred monomers b2) are the vinylesters of aliphatic C₈-C₁₈-carboxylic acids. These include the vinylesters of decanoic acid, lauric acid, myristic acid, palmitic acid, stearic acid, oleic acid and behenic acid.

Preferred monomers b3) are C₈-C₁₈-alkyl vinyl ethers. These include, for example, n-octyl, 2-ethylhexyl, n-nonyl, n-decyl, n-undecyl, n-dodecyl, n-tridecyl, n-tetradecyl, n-pentadecyl, n-hexadecyl, n-heptadecyl, n-octadecyl vinyl ether and mixtures thereof.

The cosmetic or pharmaceutical compositions according to the invention preferably comprise at least one copolymer A), which consists of copolymerized monomer units of

60 to 99% by weight of at least one N-vinyllactam and

1 to 40% by weight of at least one monomer b), as defined above.

The preparation of the copolymers A) is carried out in accordance with customary processes known to the person skilled in the art, such as, for example, solution polymerization, precipitation polymerization, emulsion polymerization or inverse suspension polymerization.

The polymerization temperatures are preferably in a range from about 30 to 200° C., particularly preferably 40 to 110° C. Suitable initiators are, for example, azo and peroxy compounds, and the customary redox initiator systems, such as combinations of hydrogen peroxide and reducing compounds, e.g. sodium sulfite, sodium bisulfite, sodium formaldehyde, sulfoxilate and hydrazine.

The K values of the copolymers A) are preferably at least 20. They are particularly preferably in a range from 20 to 100, in particular 20 to 80. The K values are determined in accordance with H. Fikentscher, Cellulosechemie [Cellulose chemistry], Volume 13, pp. 58-64 and 71-74 (1932) in aqueous or alcoholic solution at 25° C. and at concentrations between 0.1% and 5% depending of the K value range.

The cosmetic or pharmaceutical compositions according to the invention comprise, as component B), at least one cosmetically or pharmaceutically acceptable additive. Preference is given to compositions in which the component B) comprises at least one essentially water-insoluble (hydrophobic) compound which is liquid at 20° C. (oil or fatty component). Here, the composition according to the invention can comprise the oil or fatty component as cosmetic or pharmaceutical active ingredient, as carrier, as auxiliary or as a combination thereof. The rheological properties and the consistency of the oil- or fat-containing compositions according to the invention can generally be set in a wide range. “Modification of Theological properties” is here understood in a wide sense for the purposes of the present invention. The polymers A) used in the compositions according to the invention are generally suitable for thickening the consistency of hydrophobic compounds in a wide range. Depending on the base consistency of the hydrophobic liquid compound(s), it is generally possible to achieve flow properties from thin-liquid to solid (in the sense of “no longer flowable”) depending on the amount of polymer used. “Modification of Theological properties” is thus understood as meaning, inter alia, the increase in the viscosity of liquids, the improvement of the thixotropic properties of gels, the solidification of gels and waxes etc. The compositions according to the invention are thus preferably suitable for formulating oil- and fat-containing cosmetic and pharmaceutical products. Depending on the base consistency of the compounds of component B), the properties can generally be varied from a thin-liquid consistency to a gel-like consistency, depending on the amount of polymer A) used. Preferably, solutions of the polymer A) in oil or fat components B) are generally clear. Thus, advantageously, it is possible to color formulations, in particular cosmetic formulations, without impairment by the intrinsic color of the compositions according to the invention. Furthermore, the compositions according to the invention can be formulated in the form of opaque to clear gels.

The compositions according to the invention which comprise at least one oil or fatty component B) can be in the form either of homogeneous-phase or heterogeneous-phase formulations. “Homogeneous-phase formulations” have only a single phase irrespective of the number of constituents thereof. “Heterogeneous-phase compositions” are disperse systems of two or more immiscible components. These preferably include emulsions, such as, for example, O/W and W/O formulations which have at least one of the oil or fat components described in more detail below, and water as immiscible phases. In this connection, the polymers A) used according to the invention are generally used either in the water phase or else in the oil phase. Advantageously, the copolymers A) used according to the invention can usually be readily stirred into formulations based on oil and/or fatty components without the addition of solvents. In this connection, it may be advantageous to warm the components to temperatures >40° C. for formulation.

The compositions according to the invention preferably comprise the polymer component A) in an amount of about 0.001 to 50% by weight, particularly preferably 0.01 to 30% by weight, in particular 0.1 to 20% by weight, based on the total weight of the composition.

If the compositions according to the invention comprise at least one hydrophobic component B), this is preferably chosen from

i) oils,

ii) fats,

iii) waxes,

iv) esters of C₆-C₃₀-monocarboxylic acids with mono-, di- or trihydric alcohols which are different from i) to iii),

v) saturated acyclic and cyclic hydrocarbons,

vi) fatty acids,

vii) fatty alcohols,

and mixtures thereof.

The compositions according to the invention have, for example, an oil or fat component B) chosen from: hydrocarbons of low polarity, such as mineral oils; linear saturated hydrocarbons, preferably having more than 8 carbon atoms, such as tetradecane, hexadecane, octadecane etc.; cyclic hydrocarbons, such as decahydronaphthalene; branched hydrocarbons, animal and vegetable oils; waxes; wax esters; vaseline; esters, preferably esters of fatty acids, such as, for example, the esters of C₁-C₂₄-monoalcohols with C₁-C₂₂-monocarboxylic acids, such as isopropyl isostearate, n-propyl myristate, isopropyl myristate, n-propyl palmitate, isopropyl palmitate, hexacosanyl palmitate, octacosanyl palmitate, triacontanyl palmitate, dotriacontanyl palmitate, tetratriacontanyl palmitate, hexacosanyl stearate, octacosanyl stearate, triacontanyl stearate, dotriacontanyl stearate, tetratriacontanyl stearate; salicylates, such as C₁-C₁₀-salicylates, e.g. octyl salicylate; benzoate esters, such as C₁₀-C₁₅-alkyl benzoates, benzyl benzoate; other cosmetic esters, such as fatty acid triglycerides, propylene glycol onolaurate, polyethylene glycol monolaurate, C₁₀-C₁₅-alkyl lactates, etc. and mixtures thereof.

Suitable silicone oils B) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol. Preferred cyclic siloxanes have 4- to 8-membered rings. Suitable cyclic siloxanes are, for example, obtainable commercially under the name cyclomethicone.

Preferred oil and fat components B) are chosen from paraffin and paraffin oils; vaseline; natural fats and oils, such as castor oil, soybean oil, groundnut oil, olive oil, sunflower oil, sesame oil, avocado oil, cocobutter, almond oil, peach kernel oil, castor oil, cod-liver oil, lard, spermaceti, spermaceti oil, sperm oil, wheat germ oil, macadamia nut oil, evening primrose oil, Jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti, and mixtures of the abovementioned oil and fatty components.

Suitable cosmetically and pharmaceutically compatible oil and fatty components B) are described in Karl-Heinz Schrader, Grundlagen und Rezepturen der Kosmetika [Cosmetic bases and formulations], 2nd Edition, Verlag Huthig, Heidelberg, pp. 319-355, to which reference is made here.

Suitable cosmetically and/or dermatologically active ingredients B1) are, for example, tanning active ingredients, skin and hair pigmentation agents, tinting agents, coloring agents, bleaches, keratin-hardening substances, antimicrobial active ingredients, light filter active ingredients, repellent active ingredients, substances with hyperemic activity, substances with keratolytic and keratoplastic activity, antidandruff active ingredients, antiphlogistics, substances which have a keratinizing action, substances which act as antioxidants or as free-radical scavengers, skin moisturizers or humectants, refatting active ingredients, antierythimatous or antiallergic active ingredients and mixtures thereof.

Tanning active ingredients B1) which tan the skin artificially and which are suitable for tanning the skin without natural or artificial irradiation with UV rays are, for example, dihydroxyacetone, alloxan and walnut shell extract. Suitable keratin-hardening substances are usually active ingredients as are also used in antiperspirants, such as, for example, potassium aluminum sulfate, aluminum hydroxychloride, aluminum lactate, etc. Antimicrobial active ingredients B1) are used in order to destroy microorganisms or to inhibit their growth and thus serve both as preservatives and also as a deodorizing substance which prevents the formation or the intensity of body odor. These include, for example, customary preservatives known to the person skilled in the art, such as p-hydroxybenzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable light filter active ingredients B1) are substances which absorb UV rays in the UV-B- and/or UV-A region. Suitable UV filters are, for example, 2,4,6-triaryl-1,3,5-triazines in which the aryl groups may each carry at least one substituent which is preferably chosen from hydroxyl, alkoxy, specifically methoxy, alkoxycarbonyl, specifically methoxycarbonyl and ethoxycarbonyl and mixtures thereof. Also suitable are p-aminobenzoates, cinnamates, benzophenones, camphor derivatives, and pigments which stop UV rays, such as titanium dioxide, talc and zinc oxide. Suitable repellent active ingredients B1) are compounds which are able to drive away or expel certain animals, in particular insects, from humans. These include, for example, 2-ethyl-1,3-hexanediol, N,N-diethyl-m-toluamide etc. Suitable substances with hyperemic activity B1), which stimulate blood flow through the skin are, for example, essential oils, such as dwarf pine, lavender, rosemary, juniper berry, horsechestnut extract, birch leaf extract, hayseed extract, ethyl acetate, camphor, menthol, peppermint oil, rosemary extract, eucalyptus oil, etc. Suitable keratolytic and keratoplastic substances B1) are, for example, salicylic acid, potassium thioglycolate, thioglycol acid and salts thereof, sulfur, etc. Suitable antidandruff active ingredients B1) are, for example, sulfur, sulfur polyethylene glycol sorbitan monooleate, sulfur, ricinol polyethoxylate, zinc pyrithione, aluminum pyrithione, etc. Suitable antiphlogistic B1), which counter skin irritations, are, for example, allantoin, bisabolol, Dragosantol, chamomile extract, panthenol, etc.

The cosmetic compositions according to the invention can comprise, as cosmetic and/or pharmaceutical active ingredient B1) (and also optionally as auxiliary B3)), at least one cosmetically or pharmaceutically acceptable polymer different from compounds of component A). Very generally, these include anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and optionally further vinylesters (e.g. Luviset® grades), maleic anhydride copolymers, optionally reacted with alcohol, anionic polysiloxanes, e.g. carboxyfunctional, t-butyl acrylate, methacrylic acid (e.g. Luviskol® VBM), copolymers of acrylic acid and methacrylic acid with hydrophobic monomers, such as, for example, C₄-C₃₀-alkylesters of meth(acrylic acid), C₄-C₃₀-alkyl vinyl esters, C₄-C₃₀-alkyl vinyl ethers and hyaluronic acid. Examples of anionic polymers are also vinyl acetate/crotonic acid copolymers, as are available commercially, for example, under the names Resyn® (National Starch) and Gafset® (GAF), and vinylpyrrolidone/vinyl acrylate copolymers, obtainable, for example, under the trade name Luviflex® (BASF). Other suitable polymers are the vinylpyrrolidone/acrylate terpolymer available under the name Luviflex® VBM-35 (BASF), and sodium sulfonate-containing polyamides or sodium sulfonate-containing polyesters.

Further suitable polymers are cationic polymers with the INCI name Polyquaternium, e.g. copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamidocopolymers (Polyquaternium-7) and chitosan. Suitable cationic (quaternized) polymers are also Merquat® (polymer based on dimethyldiallylammonium chloride), Gafquat® (quaternary polymers which are formed by the reaction of polyvinylpyrrolidone with quaternary ammonium compounds), Polymer JR (hydroxyethylcellulose with cationic groups) and vegetable-based cationic polymers, e.g. guar polymers, such as the Jaguar® grades from Rhodia.

Further suitable polymers are also neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives.

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinylesters, such as vinyl acetate, e.g. Luviskol® VA 37 (BASF); polyamides, e.g. those based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers as are disclosed, for example, in the German patent applications DE 39 29 973, DE 21 50 557, DE 28 17 369 and DE 37 08 451. Arylamidopropyltrimethylammonium chloride/acrylic acid or methacrylic acid copolymers and the alkali metal and ammonium salts thereof are preferred zwitterionic polymers. Other suitable zwitterionic polymers are methacroylethylbetaine/methacrylate copolymers, which are available commercially under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or -dispersible polymers, e.g. polyether siloxanes, such as Tegopren® (Goldschmidt) or Belsil® (Wacker).

The carrier component B2) is preferably chosen from water, hydrophilic components, hydrophobic components and mixtures thereof.

Suitable hydrophilic carriers B2) are, for example, mono-, di- or polyhydric alcohols having, preferably, 1 to 8 carbon atoms, such as ethanol, n-propanol, isopropanol, propylene glycol, glycerol, sorbitol, etc.

Suitable hydrophobic carriers B2) are, for example, the abovementioned oil and fat components, to which reference is made here.

The cosmetically or pharmaceutically active compositions according to the invention can also comprise at least one auxiliary B3).

The formulation base of pharmaceutical compositions according to the invention preferably comprises pharmaceutically acceptable auxiliaries. Pharmaceutically acceptable auxiliaries are the auxiliaries which are known for use in the fields of pharmacy, food technology and related fields, in particular the auxiliaries listed in the relevant pharmacopoeia (e.g. DAB Ph. Eur. BP NF), and other auxiliaries whose properties do not preclude a physiological application.

Suitable auxiliaries B3) may be: lubricants, wetting agents, emulsifying and suspending agents, preservatives, antioxidants, antierythritive substances, chelating agents, emulsion stabilizers, film formers, gel formers, odor-masking agents, resins, hydrocolloids, solvents, solubility promoters, neutralizing agents, permeation accelerators, pigments, quaternary ammonium compounds, refatting and superfatting agents, ointment bases, cream bases or oil bases, silicone derivatives, stabilizers, sterilizing agents, propellants, drying agents, opacifiers, thickeners, waxes, softeners, white oils. Formulation in this regard is based on expert knowledge, as given, for example, in Fiedler, H. P. Lexikon der Hilfsstoffe für Pharmazie, Kosmetik und angrenzende Gebiete [Lexikon of auxiliaries for pharmacies, cosmetics and related fields], 4th Ed., Aulendorf: ECV-Editio-Kantor-Verlag, 1996.

To prepare the dermatological compositions according to the invention, the active ingredients can be mixed or diluted with a suitable auxiliary (excipient). Excipients can be solid, semisolid or liquid materials which can also serve as vehicles, carriers or medium for the active ingredient. The admixing of further auxiliaries is carried out, where desired, in the manner known to the person skilled in the art.

Examples of preferred cosmetic and dermatological formulations are ointments, creams, emulsions, suspensions, lotions, milks, pastes, gels, foams, sprays, etc. If desired, liposomes or microspheres can also be used.

The cosmetic compositions according to the invention may be skin cosmetic, dermatological or hair cosmetic compositions.

In a first preferred embodiment, the compositions according to the invention are skin-cleansing compositions.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower bath and shower gels, foam baths, oil baths and scrub preparations.

According to a further preferred embodiment, the compositions according to the invention are cosmetic compositions for the care and protection of the skin, nail care compositions or preparations for decorative cosmetics.

Particular preference is given to skincare compositions, personal hygiene compositions, foot care compositions, light protection compositions, repellents, shaving compositions, depilatory compositions, antiacne compositions, make-up, mascara, lipsticks, eye shadows, kohl pencils, eyeliners, blusher and eyebrow pencils.

The skincare compositions according to the invention are, in particular, W/O or O/W skin creams, day creams and night creams, eye creams, face creams, antiwrinkle creams, moisturizing creams, bleaching creams, vitamin creams, skin lotions, care lotions and moisturizing lotions.

Skin cosmetic and dermatological compositions based on the above-described polymers A) exhibit advantageous effects. The polymers can, inter alia, contribute to the moisturizing and conditioning of the skin and to an improvement in the feel of the skin. The polymers can also act as thickeners in the formulations. By adding the polymers according to the invention, it is possible to achieve a considerable improvement in skin compatibility in certain formulations.

Skin cosmetic and dermatological compositions preferably comprise at least one copolymer A) in an amount of from about 0.001 to 50% by weight, preferably 0.01 to 30% by weight, very particularly preferably 0.1 to 20% by weight, based on the total weight of the composition.

Light protection agents based on the copolymers A), in particular, have the property of increasing the residence time of the UV-absorbing ingredients compared with customary auxiliaries such as polyvinylpyrrolidone.

Depending on the field of use, the compositions according to the invention can be applied in a form suitable for skin care, such as, for example, as cream, foam, gel, pencil, mousse, milk, spray (pump spray or spray containing propellant) or lotion.

As well as comprising the polymers A) and suitable carriers, the skin cosmetic preparations can also comprise further active ingredients and auxiliaries customary in skin cosmetics and as described above. These include, preferably, emulsifiers, preservatives, perfume oils, cosmetic active ingredients, such as phytantriol, vitamins A, E and C, retinol, bisabolol, panthenol, light protection agents, bleaches, colorants, tinting agents, tanning agents, collagen, protein hydrolysates, stabilizers, pH regulators, dyes, salts, thickeners, gel formers, consistency regulators, silicones, moisturizers, resetting agents and further customary additives.

Preferred oil and fatty components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with traditional polymers where specific properties are to be set.

To set certain properties, such as, for example, improving the feel to the touch, the spreading behavior, the water resistance and/or the binding of active ingredients and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, olyether siloxanes or silicone resins.

The cosmetic or dermatological preparations are prepared by customary methods known to the person skilled in the art.

The cosmetic and dermatological compositions are preferably in the form of emulsions, in particular as water-in-oil (W/O) emulsions or oil-in-water (O/W) emulsions. It is, however, also possible to choose other types of formulation, for example hydrodispersions, gels, oils, oleogels, multiple emulsions, for example in the form of W/O/W or O/W/O emulsions, anhydrous ointments or ointment bases, etc.

The emulsions are prepared by known methods. Apart from the copolymer A), the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The choice of emulsion type-specific additives and the preparation of suitable emulsions is described, for example, in Schrader, Grundlagen und Rezepturen der Kosmetika [Cosmetic bases and formulations], Hüthig Buch Verlag, Heidelberg, 2nd Edition, 1989, third part, to which express reference is made here.

A suitable emulsion, e.g. for a skin cream etc., generally comprises an aqueous phase which is emulsified by means of a suitable emulsifier system in an oil or fatty phase.

The proportion of the emulsifier systems in this type of emulsion is preferably about 4 to 35% by weight, based on the total weight of the emulsion. The proportion of the fatty phase is preferably about 20 to 60% by weight. The proportion of the aqueous phase is preferably about 20 to 70%, in each case based on the total weight of the emulsion. The emulsifiers are those customarily used in this type of emulsion. They are chosen, for example, from: C₁₂-C₁₈-sorbitan fatty acid esters; esters of hydroxystearic acid and C₁₂-C₃₀-fatty alcohols; mono- and diesters of C₁₂-C₁₈-fatty acids and glycerol or polyglycerol; condensates of ethylene oxide and propylene glycols; oxypropylenated/oxyethylated C₁₂-C₁₈-fatty alcohols; polycyclic alcohols, such as sterols; aliphatic alcohols with a high molecular weight, such as lanolin; mixtures of oxypropylenated/polyglycerolated alcohols and magnesium isostearate; succinic esters of polyoxyethylenated or polyoxypropylenated fatty alcohols; and mixtures of magnesium lanolate, calcium lanolate, lithium lanolate, zinc lanolate or aluminum lanolate and hydrogenated lanolin or lanolin alcohol. Preferred fatty components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start-point under atmospheric pressure is at about 250° C. and whose distillation end-point is at 410° C., such as, for example, vaseline oil; esters of saturated or unsaturated fatty acids, such as alkyl myristates, e.g. i-propyl, butyl or cetyl myristate, hexadecylstearate, ethyl or i-propyl palmitate, octanoic or decanoic acid triglycerides and cetyl ricinoleate.

The fatty phase may also comprise silicone oils soluble in other oils, such as dimethylpolysiloxane, methylphenylpolysiloxane and the silicone glycol copolymer, fatty acids and fatty alcohols.

In order to favor the retention of oils, in addition to the polymers A), it is also possible to use waxes, such as, for example, carnauba wax, candililla wax, beeswax, microcrystalline wax, ozokerite wax and the oleates, myristates, linoleates and stearates of Ca, Mg and Al.

The water-in-oil emulsions are generally prepared by introducing the fatty phase and the emulsifier into a reaction vessel. The vessel is heated at a temperature of approximately 50 to 75° C., then the active ingredients and/or auxiliaries which are soluble in oil are added, and water which has been heated beforehand to approximately the same temperature and into which the water-soluble ingredients have optionally been dissolved beforehand is added with stirring. The mixture is stirred until an emulsion of the desired fineness is achieved, which is then left to cool to room temperature, if necessary with a lesser amount of stirring.

According to a further preferred embodiment, the compositions according to the invention are a shower gel, a shampoo formulation or a bath preparation.

Such formulations comprise at least one polymer A) and customary anionic surfactants as base surfactants and amphoteric and/or nonionic surfactants as cosurfactants. Further suitable active ingredients and/or auxiliaries are generally chosen from lipids, perfume oils, dyes, organic acids, preservatives and antioxidants, and thickener/gel formers, skin conditioning agents and humectants.

These formulations preferably comprise 2 to 50% by weight, preferably 5 to 40% by weight particularly preferably 8 to 30% by weight, of surfactants, based on the total weight of the formulation.

All anionic, neutral, amphoteric or cationic surfactants customarily used in body-cleansing compositions can be used in washing, shower and bath preparations.

Suitable anionic surfactants are, for example, alkyl sulfates, alkyl ether sulfates, alkylsulfonates, alkylarylsulfonates, alkyl succinates, alkyl sulfosuccinates, N-alkylsarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkylamphoacetates or amphopropionates, alkyl amphodiacetates or amphodipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mol per mole of alcohol. Also suitable are alkylamine oxides, mono- or dialkylalkanolamides, fatty acid esters of polyethylene glycols, ethoxylated fatty acid amides, alkyl polyglycosides or sorbitan ether esters.

The washing, shower and bath preparations can also comprise customary cationic surfactants such as, for example, quaternary ammonium compounds, for example cetyltrimethylammonium chloride.

In addition, it is also possible to use other customary cationic polymers, such as, for example, copolymers of acrylamide and dimethyldiallylammonium chloride (Polyquaternium-7), cationic cellulose derivatives (Polyquaternium-4, -10), guar hydroxypropyltrimethylammonium chloride (INCI: Hydroxylpropyl Guar Hydroxypropyltrimonium Chloride), copolymers of N-vinylpyrrolidone and quaternized N-vinylimidazole (Polyquaternium-16, -44, -46), copolymers of N-vinylpyrrolidone/dimethylaminoethyl methacrylate, quaternized with diethyl sulfate (Polyquaternium-11) and others.

The shower gel/shampoo formulations can further comprise thickeners, such as, for example sodium chloride, PEG-55, propylene glycol oleate, PEG-120 methyl glucose dioleate and others, and also preservatives, further active ingredients and auxiliaries and water.

In a further preferred embodiment the compositions according to the invention are hair-treatment compositions.

Hair-treatment compositions according to the invention preferably comprise at least one copolymer A) in an amount in the range from about 0.1 to 30% by weight, preferably 0.5 to 20% by weight, based on the total weight of the composition.

The hair-treatment compositions according to the invention are preferably in the form of a setting foam, hair mousse, hair gel, shampoo, hairspray or hair foam. Hairsprays include both aerosol sprays and also pump sprays without propellant gas. Hair foams include both aerosol foams and also pump foams without propellant gas.

Hairsprays and hair foams preferably comprise predominantly or exclusively water-soluble or water-dispersible components. If the compounds used in the hairsprays and hair foams according to the invention are water-dispersible, they can be used in the form of aqueous microdispersions with particle diameters of, usually, 1 to 350 nm, preferably 1 to 250 nm. The solids contents of these preparations are customarily in a range from about 0.5 to 20% by weight. These microdispersions generally do not require emulsifiers or surfactants for their stabilization.

Preferred hair-treatment compositions are in the form of an aqueous dispersion or in the form of an alcoholic or aqueous-alcoholic solution. Examples of suitable alcohols are ethanol, propanol, isopropanol and mixtures thereof.

Furthermore, the hair-treatment compositions according to the invention can generally comprise customary cosmetic auxiliaries, for example softeners, such as glycerol and glycol; emollients; perfumes; surfactants; UV absorbers; dyes; antistatic agents; agents for improving combability; preservatives; and antifoams.

If the compositions according to the invention are formulated as hairspray, they comprise a sufficient amount of a propellant, for example a low-boiling hydrocarbon or ether, such as propane, butane, isobutane or dimethyl ether. Propellants which can be used are also compressed gases, such as nitrogen, air or carbon dioxide. The amount of propellant here can be kept low in order not to increase the VOC content unnecessarily. This is then generally not more than 55% by weight, based on the total weight of the composition. If desired, however, higher VOC contents of 85% by weight and above are also possible.

The above-described polymers A) can also be used in combination with other hair polymers in the compositions. Suitable polymers are those described above.

The other hair polymers are preferably present in amounts up to. 10% by weight, based on the total weight of the composition.

A preferred hair-treatment composition comprises:

a) 0.5 to 20% by weight, preferably 1 to 10% by weight, of at least one polymer A), as defined above,

b) 50 to 99.5% by weight, preferably 55 to 99% by weight, of a carrier (solvent), chosen from water and water-miscible solvents, preferably C₂-C₅-alcohols, in particular ethanol, and mixtures thereof,

c) 0 to 70% by weight, preferably 0.1 to 50% by weight, of a propellant, preferably chosen from dimethyl ether and alkanes, such as, for example, propane/butane mixtures,

d) 0 to 10% by weight, preferably 0.1 to 10% by weight, of at least one hair polymer different from a), preferably a water-soluble or -dispersible polymers

e) 0 to 0.5% by weight, preferably 0.001 to 2% by weight, of at least one water-soluble or water-dispersible silicone compound,

and optionally further active ingredients and/or auxiliaries, as defined above.

The composition according to the invention can comprise, as component e), at least one nonionic, siloxane-containing, water-soluble or -dispersible polymer, in particular chosen from the above-described polyether siloxanes. The proportion of this component is then generally about 0.001 to 2% by weight, based on the total weight of the composition.

The composition according to the invention can comprise, as additional component, at least one water-insoluble silicone, in particular a polydimethylsiloxane, e.g. the Abil® grades from Goldschmidt. The proportion of this component is then generally about 0.0001 to 0.2% by weight, preferably 0.001 to 0.1% by weight, based on the total weight of the composition.

The composition according to the invention can additionally optionally comprise an antifoam, e.g. one based on silicone. The amount of antifoam is generally up to about 0.001% by weight, based on the total amount of the composition.

The invention further provides for the use of at least one copolymer which comprises

a) 60 to 99% by weight of at least one hydrophilic monomer and, of this, at least 65% by weight comprises at least one N-vinyllactam, and

b) 1 to 40% by weight of at least one hydrophobic monomer chosen from

b1) compounds of the formula I

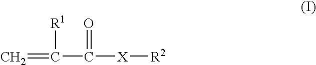

in which

R¹ is H, methyl or ethyl,

X is O, NH or NR³,

R² is C₈-C₃₀-alkyl, C₈-C₃₀-alkenyl, cycloalkyl-C₆-C₂₂-alkyl, cycloalkyl-C₆-C₂₂-alkenyl, aryl-C₆-C₂₂-alkyl or aryl-C₆-C₂₂-alkenyl, and

R³ is C₁-C₇-alkyl, cycloalkyl, aryl or one of the meanings given for R²,

b2) vinyl esters of aliphatic C₈-C₃₀-carboxylic acids,

b3) C₈-C₃₀-alkyl vinyl ethers

and mixtures thereof,

in copolymerized form, as component of a homogeneous-phase or heterogeneous-phase cosmetic or pharmaceutical composition which comprises at least one hydrophobic component, for modifying the Theological properties of this composition.

The invention further provides for the use of a copolymer, as defined above, in skin-cleansing compositions, compositions for the care and protection of the skin, nail care compositions, preparations for decorative cosmetics, shower gels, shampoos, bath preparations and hair-treatment compositions.

The invention further provides for the use of the copolymers A) in skin-cleansing compositions, compositions for the care and protection of the skin, nail care compositions, preparations for decorative cosmetics, shower gels, shampoos and bath preparations which comprise water and at least one hydrophobic component, for moisturizing the skin, for conditioning the skin, i.e. e.g. for improving the feel by touch and the smoothness, as thickeners, and/or for improving skin compatibility, i.e. for reducing irritancy, specifically toward surfactants, and also for achieving better wetting.

In hair-treatment compositions, the copolymers A) are preferably used as setting agents and/or conditioners. The composition here is preferably in the form of a setting foam, hair mousse, hair gel, shampoo, hairspray or hair foam.

The invention is illustrated in more detail by reference to the following, nonlimiting examples.

### EXAMPLES

### I. Application Examples

### Application Example 1

### Skin Cream

A water/oil cream emulsion (skin cream A) was prepared according to the following formulation:

| CTFA name | | % by wt. |
|---|---|---|
| Cremophor ® A6 (BASF | Ceteareth-6 (stearyl alcohol | 2.0 |
| AG) | ethoxylate) and stearyl | |
| | alcohol | |
| Cremophor ® A25 (BASF | Ceteareth-25 (fatty alcohol | 2.0 |
| AG) | ethoxylate) | |
| Lanette ® | Cetearyl alcohol | 2.0 |
| Imwitor ® 960 K | Glyceryl stearate SE | 3.0 |
| Paraffin oil | | 5.0 |
| Jojoba oil | | 4.0 |
| Luvitol ® EHO (BASF AG) | Cetearyl octanoate (cetyl- | 3.0 |
| | stearyl 2-ethylhexanoate) | |
| ABIL ® 350 (Goldschmidt) | Dimethicone (polydimethyl- | 1.0 |
| | siloxane) | |
| Amerchol ® L101 (Amerchol | Mineral oil and lanolin | 3.0 |
| Edison) | alcohol | |
| Veegum Ultra | Magnesium aluminum silicate | 0.5 |
| 1,2-Propylene glycol | Propylene glycol | 5.0 |
| Abiol | Imidazolindinylurea | 0.3 |
| Phenoxyethanol | | 0.5 |
| D-Panthenol USP | | 1.0 |

| | |
|---|---|
| Vinylpyrrolidone/stearyl methacrylate polymer, 70/30% | 0.5 |
| by wt. (K value 50; 1% in isopropanol) | |
| Water | ad 100 |

### Application Example 2

### Shower Gel/Shampoo

A shower gel formulation (shower gel A) according to the invention was firstly prepared according to the following formulation:

| CTFA name | | % by wt. |
|---|---|---|
| Texapon ® NSO (Henkel) | Sodium laureth sulfate | 40.0 |
| | (sodium lauryl ether | |
| | sulfate) | |
| Tego Betain ® L7 (Goldschmidt) | Cocamidopropylbetaine | 5.0 |
| Plantacare 2000 | Decyl glucoside | 5.0 |
| Perfume | | 0.2 |
| Vinylpyrrolidone/stearyl methacrylate polymer, 70/30% | | 0.2 |
| by wt. (K value 50; 1% in isopropanol) | | |
| Euxyl K ® 100 (Schulke & | Benzyl alcohol, methyl- | 0.1 |
| Mayr) | chloroisothiazolinone, | |
| | methylisothiazolinone | |
| D-Panthenol USP | | 0.5 |
| Citric acid (pH 6-7) | | q.s. |
| NaCl | | 2.0 |
| Water | | ad 100 |

Three comparison shower gels were prepared in the same way, specifically:

Shower gel B: (copolymer according to the invention replaced by equal amount of the noncrosslinked polymer Polyquaternium-16)

Shower gel C: (copolymer according to the invention replaced by equal amount of cationically modified hydroxyethylcellulose)

Shower gel D: (without the addition of polymer)

The following comparison test was carried out with shower gels A, B, C and D to determine the foam creaminess:

In each case, 2.0 g of the abovementioned formulation were placed onto the palm of the left hand and lathered with tap water, and after rubbing for 1 minute between both hands, the foam feeling in the palms of the hands was assessed:

Grade 1: very creamy

Grade 2: creamy

Grade 3: dull/lacking substance

Result of the Comparative Test (Average Value of Grading by 10 Subjects)

| Shower gel | Average value from 10 subjects |
|---|---|
| A | 1.4 |
| B | 2.9 |
| C | 2.0 |
| D | 3.0 |

### Application Example 3

### Moisturizing formulation Formulation A

| CTFA name | | % by wt. |
|---|---|---|
| Cremophor ® A6 (BASF AG) | Ceteareth-6 (stearyl | 2.0 |
| | alcohol ethoxylate) and | |
| | stearyl alcohol | |
| Cremophor ® A25 (BASF AG) | Ceteareth-25 (fatty alcohol | 2.0 |
| | ethoxylate) | |
| Paraffin oil | | 10 |
| Lanette ® O | Cetearyl alcohol | 2.0 |
| Stearic acid | | 3.0 |
| Nip-Nip (Nipa Laboratories | Methyl and propyl | 0.5 |
| Ltd., USA) | 4-hydroxybenzoate (7:3) | |
| Abiol | Imidazolindinylurea | 0.5 |

| | |
|---|---|
| Vinylpyrrolidone/stearyl methacrylate polymer, 80/20% | 3.0 |
| by wt. (K value 25; 1% in isopropanol) | |
| Water | ad 100 |

To prepare the formulation, the two phases were heated to 80° C., phase a) and b) were stirred, homogenized and stirred until cold, and then 10% strength aqueous NaOH solution was used to adjust the pH to 6.

In the same way a comparison cream (formulation B) was prepared without the addition of polymer.

A subject test on 8 subjects was carried out using formulations A and B. For this, the formulations were each applied to the forearm of the subjects in an amount of 2 mg/cm². After 30 min, the moisture content of the skin was determined using a Corneometer CM 825 (Khazaka & Courage). Following the application of formulation A, an average volume of 41 Corneometer units was measured, and with formulation B the average value was 35.

### Application Example 4

### O/W Cream for Moisturizing Skin

| Additive | % by wt. |
|---|---|
| Glycerol monostearate | 2.0 |
| Cetyl alcohol | 3.0 |
| Paraffin oil, subliquidum | 15.0 |
| Vaseline | 3.0 |
| Caprylic/capric triglyceride | 4.0 |
| Octyldodecanol | 2.0 |
| Hydrogenated coconut fat | 2.0 |
| Cetyl phosphate | 0.4 |
| Vinylpyrrolidone/acrylic acid/stearyl methacrylate | 3.0 |
| polymer, 60/5/35% by wt. (K value 41; 1% in isopropanol) | |
| Glycerol | 3.0 |
| Sodium hydroxide | q.s. |
| Perfume oil | q.s. |
| Preservative | q.s. |
| Water | ad 100 |

### Application Example 5

### O/W Lotion

| Additive | % by wt. |
|---|---|
| Stearic acid | 1.5 |
| Sorbitan monostearate | 1.0 |
| Sorbitan monooleate | 1.0 |
| Paraffin oil, subliquidum | 7.0 |
| Cetyl alcohol | 1.0 |
| Polydimethylsiloxane | 1.5 |
| Glycerol | 3.0 |
| Vinylpyrrolidone/acrylic acid/stearyl methacrylate | 0.5 |
| polymer, 50/10/40% by wt. (K value 36; 1% in isopropanol) | |
| Perfume oil | q.s. |
| Preservative | q.s. |
| Water | ad 100 |

### Application Example 6

### W/O Lotion

| Additive | % by wt. |
|---|---|
| PEG-7 hydrogenated castor oil | 4.0 |
| Wool wax alcohol | 1.5 |
| Beeswax | 3.0 |
| Triglyceride, liquid | 5.0 |
| Vaseline | 9.0 |
| Ozokerite | 4.0 |
| Paraffin oil, subliquidum | 4.0 |
| Glycerol | 2.0 |
| Vinylpyrrolidone/acrylic acid/stearyl methacrylate | 10.0 |
| polymer, 80/10/10% by wt. (K value 45; 1% in isopropanol) | |
| Magnesium sulfate*7H₂O | 0.7 |
| Perfume oil | q.s. |
| Preservative | q.s. |
| Water | ad 100 |

### Application Example 7

### Hydrogel for Skincare

| Additive | % by wt. |
|---|---|
| Vinylpyrrolidone/acrylic acid/stearyl methacrylate | 20.0 |
| polymer, 50/20/30% by wt. (K value 36; 1% in isopropanol) | |
| Sorbitol | 2.0 |
| Glycerol | 3.0 |
| Polyethylene glycol 400 | 5.0 |
| Ethanol | 1.0 |
| Perfume oil | q.s. |
| Preservative | q.s. |
| Water | ad 100 |

### Application Example 8

### Liquid Soap

| Additive | % by wt. |
|---|---|
| Coconut fatty acid, potassium salt | 15 |
| Potassium oleate | 3 |
| Glycerol | 5 |
| Vinylpyrrolidone/stearyl methacrylate polymer, | 2 |
| 70/30% by wt. (K value 47; 1% in isopropanol) | |
| Glycerol stearate | 1 |
| Ethylene glycol distearate | 2 |
| Specific additives, complexing agents, fragrances, | ad 100 |
| water | |

### Application Example 9

### W/O Sunscreen Emulsion

| Additive | % by wt. |
|---|---|
| Magnesium stearate | 0.5 |
| Aluminum stearate | 0.5 |
| Hydrogentated castor oil, 7 EO | 6.0 |
| Vinylpyrrolidone/stearyl methacrylate polymer, | 5.0 |
| 70/30% by wt. (K value 47; 1% in isopropanol) | |
| Microwax | 1.0 |
| Cetylstearyl 2-ethylhexanoate | 10.0 |
| 2-Hydroxy-4-methoxybenzophenone | 2.0 |
| Polyethoxyethyl 4-bis(polyethoxy)aminobenzoate | 5.0 |
| α-Bisabolol | 0.2 |
| 1,2-Propylene glycol | 3.0 |
| Perfume oil | q.s. |
| Preservative | q.s. |
| Water | ad 100 |

### Application Example 10

### Sunscreen Oil

| Additive | % by wt. |
|---|---|
| vinylpyrrolidone/stearyl methacrylate polymer, | 10.0 |
| 70/30% by wt. (K value 45; 1% in isopropanol) | |
| 3-(4′-Methyl)benzylidenebornan-2-one | 2.5 |
| 2-Hydroxy-4-methoxybenzophenone | 5.5 |
| Caprylic/capric triglyceride | 10.0 |
| Dibutyl adipate | 22.5 |
| Isopropyl myristate | 7.5 |
| Paraffin oil | 42.0 |

We claim:

1. A cosmetic composition with a liquid to gel-like consistency, comprising:
A) at least one copolymer which consists of, based on the total amount of monomer, copolymerized monomer units of
a) 60 to 99% by weight of at least one N-vinyllactam, and
b) 1 to 40% by weight of at least one hydrophobic monomer chosen from
compounds b1) of the formula I 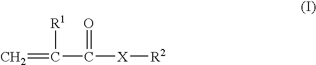
in which
R¹ is H, methyl or ethyl,
X is O, NH or NR³,
R² is C₈-C₃₀-alkyl, C₈-C₃₀-alkenyl, cycloalkyl-C₂-C₂₂-alkyl, cycloalkyl-C₂-C₂₂-alkenyl, aryl-C₂-C₂₂-alkyl or aryl-C₂-C₂₂-alkenyl, and
R³ is C₁-C₇-alkyl, cycloalkyl, aryl or one of the meanings given for R²
and
B) at least one cosmetically acceptable additive.

2. A composition as claimed in claim 1, where the N-vinyllactam is chosen from N-vinylpyrrolidone, N-vinylpiperidone, N-vinylcaprolactam and mixtures thereof.

3. A liquid composition as claimed in claim 1 with a viscosity ranging from 1 to 6000 mPas.

4. A gel-like composition as claimed in claim 1 with a viscosity ranging from more than 600 to 60000 mPas.

5. A composition as claimed in any of the preceding claims, where the component B) comprises at least one hydrophobic compound liquid at 20° C.

6. A composition as claimed in any of the preceding claims, where the component B) is chosen from
B1) cosmetic and pharmaceutical active ingredients,
B2) carriers and
B3) auxiliaries.

7. A composition as claimed in claim 6, where the component B2) is chosen from water, hydrophilic components, hydrophobic components and mixtures thereof.

8. A composition as claimed in any of claims 5 to 7, comprising at least one hydrophobic component chosen from
i) oils,
ii) fats,
iii) waxes,
iv) esters of C₆-C₃₀-monocarboxylic acids with mono-, di- or trihydric alcohols which are different from i) to iii),
v) saturated acyclic and cyclic hydrocarbons,
vi) fatty acids,
vii) fatty alcohols,
and mixtures thereof.

9. A composition as claimed in any of the preceding claims in the form of an ointment, cream, emulsion, suspension, lotion, milk, paste, a gel, foam or spray.

10. A hair-treatment composition comprising:
a) 0.5 to 20% by weight of at least polymer A), as defined in one of claims 1 to 2,
b) 50 to 99.5% by weight of a carrier chosen from water, water-miscible solvents, preferably C₂- to C₅-alcohols, and mixtures thereof,
c) 0 to 70% by weight of a propellant which is preferably chosen from dimethyl ether and alkanes,
d) 0 to 10% by weight of at least one hair polymer different from a),
e) 0 to 0.5% by weight of at least one water-soluble or water-dispersible silicone compound,
and optionally further active ingredients and/or auxiliaries.

11. The use of at least one copolymer which consists of copolymerized monomer units of
a) 60 to 99% by weight of at least one N-vinyllactam, and
b) 1 to 40% by weight of at least one hydrophobic monomer chosen from
b1) compounds of the formula I 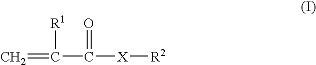
in which
R¹ is H, methyl or ethyl,
X is O, NH or NR³,
R² is C₈-C₃₀-alkyl, C₈-C₃₀-alkenyl, cycloalkyl-C₆-C₂₂-alkyl, cycloalkyl-C₆-C₂₂-alkenyl, aryl-C₆-C₂₂-alkyl or aryl-C₆-C₂₂-alkenyl, and
R³ is C₁-C₇-alkyl, cycloalkyl, aryl or one of the meanings given for R²,
b2) vinyl esters of aliphatic C₈-C₃₀-carboxylic acids,
b3) C₈-C₃₀-alkyl vinyl ethers
and mixtures thereof,
as component of a homogeneous-phase or heterogeneous-phase cosmetic composition which comprises at least one hydrophobic component, for modifying the rheological properties of this composition.

12. The use of a copolymer as defined in claim 11 in skin-cleansing compositions, compositions for the care and protection of the skin, nail care compositions, preparations for decorative cosmetics, shower gels, shampoos, bath preparations and hair-treatment compositions.

13. The use as claimed in claim 12 in skin-cleansing compositions, compositions for the care and protection of the skin, nail care compositions, preparations for decorative cosmetics, shower gels, shampoos and bath preparations which comprise water and at least one hydrophobic component, for moisturizing the skin, for conditioning the skin, as thickeners, for improving skin compatibility and/or for achieving better wetting.

14. The use as claimed in claim 12 in hair-treatment compositions as setting agents and/or conditioners.

15. The use as claimed in claim 14, where the composition is in the form of a setting foam, hair mousse, hair gel, shampoo, hairspray or hair foam.
